(19) United States
(12) Patent Application Publication
(10) Pub. No.: US 2026/0027167 A1
(43) Pub. Date: Jan. 29, 2026

(54) COMPOSITION COMPRISING FERMENTED CEREAL GRUEL

(71) Applicant: NORDIC REBALANCE A/S, Hillerød (DK)

(72) Inventor: Hans ISRAELSEN, Allerød (DK)

(21) Appl. No.: 18/996,755

(22) PCT Filed: Jul. 18, 2023
(86) PCT No.: PCT/EP2023/069972

§ 371 Date: Jan. 17, 2025

(30) Foreign Application Priority Data
Jul. 18, 2022 (SE) 2230243-4

(51) Int. Cl. A61K 35/747; A61K 36/899

(52) U.S. Cl. CPC A61K 35/747; A61K 36/899; A61K 2236/19

(57) ABSTRACT

The present invention is directed to compositions, including post-biotic compositions. comprising cereal gruel fermented by Lactobacillus plantarum 299 (Lp299) and/or Lactobacillus plantarum 299v (Lp299v), or a related Lactobacillus cell. The compositions are useful for treating impaired gut barrier function by maintaining or restoring the gut barrier and/or the gut barrier function of an individual, including the permeability of the gut barrier.

### BACKGROUND OF THE INVENTION

The present invention is directed to compositions, including post-biotic compositions, comprising cereal gruel fermented by Lactobacillus plantarum 299 (Lp299) and/or Lactobacillus plantarum 299v (Lp299v). The compositions may be used for treating inflammation associated with or resulting from impaired gut barrier function by, e.g., maintaining or restoring the gut barrier and/or the gut barrier function of an individual, including the permeability of the gut barrier.

Chronic inflammation has been associated with a vast number of different diseases, and a better understanding of the involvement of inflammation in disease treatment is needed.

The occurrence of a chronic inflammation in an individual may have severe consequences and include elevated risks of contracting, e.g., different metabolic and cardiovascumar diseases, such as, e.g., type 2 diabetes and hypertension. Inflammation also play a role in many liver diseases.

WO 2007/036230 discloses compositions comprising cereal gruel fermented by Lactobacillus plantarum 299 (Lp299) and/or Lactobacillus plantarum 299v (Lp299v) for use in the treatment of symptoms associated with IBS, ulcerative colitis and Crohn's disease.

Lactobacillus plantarum 299v has been used in studies of gut barrier function in animal models, including rats, as reviewed by Nordström et al. (2021) in Beneficial. Microbes, vol. 12(5), pp. 441-465. Several clinical studies involving Lactobacillus plantarum 299v have generated discrepant results, and these studies are considered by Nordström et al. (2021) to be inconclusive.

While Lactobacillus plantarum 299v has been shown to affect mucin production and pathogen inhibition in the gut, Lactobacillus plantarum 299v/299 for use in treatment of inflammation associated with or resulting from impaired gut barrier function and/or for use in treatment of aberrant or impaired gut permeability-related diseases and disorders in a human being has not been described in the prior art.

Bednarska et al. (2021) describe an effect of fermented oat on the colonic mucosal barrier in individuals having irritable bowel syndrome (IBS) (Neurogastroenterology and Motility, vol. 33, Suppl. 2, EMBASE abstract EMB-636415552).

Bednarska et al. (2022) describe a possible beneficial effect of a postbiotic fermented oat gruel on the colonic mucosal barrier in patients with irritable bowel syndrome (IBS) (Front. Nutr., 9:1004084, doi:10.3389).

There is a need for novel compositions and methods for preventing or treating, including prophylactically treating, inflammation in an individual, for example a mammal, such as a human being, including inflammation associated with or resulting from an impaired gut barrier function, as well as a need for compositions and methods for preventing or treating aberrant or impaired gut permeability-related diseases and disorders in an individual, such as a mammal, including a human being. Also, there is a need for novel treatment regimens aimed at prophylactic treatment of an impaired gut barrier.

### SUMMARY OF THE INVENTION

The present invention is directed to compositions and ready-to-use products for use in treating (and prophylaxis of) inflammation in an individual, including, but not limited to, inflammation associated with or resulting from an impaired gut barrier function in the individual.

The compositions and ready-to-use products for use according to the present invention are also capable of treating diseases in which inflammation is a contributing factor to the occurrence or development of the disease.

In one aspect of the invention the compositions and ready-to-use products are capable of modulating gut permeability in an individual by maintaining, protecting, restoring and/or repairing the gut barrier and/or the gut barrier function from, e.g., physiological imbalances caused by harmful substances. Exemplary harmful substances include, e.g., pro-inflammatory and/or pathogenic microorganisms and their metabolites, as well as physiologically and environmentally toxic substances and organic compounds.

Accordingly, the compositions according to the present invention are useful in treating a number of clinical conditions, including intestinal and/or extra-intestinal inflammations, that affect, or are affected or caused by, a physiologically deficient, impaired or aberrant permeability of the gut barrier in a mammal, including a human being.

In one aspect of the invention, impaired gut barrier function is defined herein as a clinical condition or disease subject to treatment with the compositions according to the present invention, wherein said treatment alleviates or eliminates in an individual, including a human being, at least one symptom associated with said clinical condition or disease, including intestinal and/or extra-intestinal low-grade inflammation and/or chronic inflammation.

By treating impaired or aberrant gut barrier function, including impaired or aberrant gut barrier permeability, the compositions according to the present invention are also capable of
preventing or treating a number of diseases or clinical disorders related to an impaired or aberrant gut barrier function in an individual, as well as
preventing or treating impaired or aberrant gut permeability-related diseases and disorders in an individual.

Examples of impaired or aberrant gut permeability-related diseases and disorders in the gut are, e.g., gastric ulcers, infectious diarrhea, irritable bowel disease (IBS), inflammatory bowel disease (IBD), including Crohn's disease and ulcerative colitis, celiac disease and cancer, including cancer of the esophagus and colorectal cancer.

Examples of impaired or aberrant gut permeability-related extra-intestinal diseases and disorders (i.e. diseases or disorders in bodily organs other than the gut) are, e.g., allergies, auto-immune diseases, infections, inflammations, including low-grade inflammations (typically “low-grade” by e.g. defying a diagnosis), chronic inflammations, including arthritis, and acute inflammations, including sepsis, systemic inflammatory response syndrome (SIRS), multiple organ dysfunction syndrome (MODS) and multiple organ failure (MOF), as well as metabolic diseases, including metabolic syndrome associated with one or more of abdominal obesity, high blood pressure, impaired fasting glucose, high triglyceride levels, and low HDL cholesterol levels, as well as other types of obesity-associated metabolic diseases, including diabetes (of either type I of Type II), and cardiovascular diseases.

An aberrant or impaired gut barrier permeability may be associated with inflammation of the intestinal cell wall, including mucosal and submucosal tissues thereof. However, gut inflammation may also occur in the absence of an aberrant or impaired gut permeability in an individual. The claimed compositions are also capable of treating an inflammation of the intestinal cell wall, including mucosal and submucosal tissues thereof, including acute, low-grade or chronic inflammation.

Treating or ameliorating inflammation and protecting, maintaining or restoring gut barrier permeability to a physiologically healthy status in an individual are essential elements in treatment of diseases, such as, e.g., irritable bowel syndrome (IBS), inflammatory bowel disease (IBD), including Crohn's disease and ulcerative colitis, as well as, e.g., type I diabetes, type II diabetes, obesity, rheumatoid arthritis and multiple sclerosis.

Restoring or protecting the gut barrier improves or maintains physiologically healthy levels of digestion and absorption of nutrients across the gut barrier without concomitant translocation of the gut barrier of harmful substances that will eventually cause, e.g., low-grade and/or chronic inflammation either systemically, in the gut, or in one or more extra-intestinal organs of the body of an individual, including a human being.

The gut mucosa comprises epithelial cells, which secrete mucus in the form of a thick protective fluid. The epithelial cells receive signals from both the lumen of the gut and from the basolateral side of the gut. On the basolateral side of the gut, the epithelial cells receive signals from different types of cells, including immune cells, nerve cells and stromal cells. Accordingly, gut barrier integrity and homeostasis of the gut barrier involve epithelial cell signalling, and the compositions according to the present invention are capable of maintaining or restoring gut barrier integrity and homeostasis.

Epithelial cells in the gut barrier are held together by junctional protein complexes comprising tight junctions (TJ) and adherens junctions (AJ). Tight junctions (TJs) are also termed occluding junctions, or zonulae occludentes. TJs are multiprotein junctional complexes with a canonical function that prevent leakage of solutes and water by forming seals between epithelial cells.

Adherens junctions (AJs) are cell-cell adhesion complexes that are continuously assembled and disassembled, thereby allowing epithelial cells of the gut to respond to forces, biochemical signals and structural changes in the gut. The compositions according to the present invention are believed to be capable of restoring and/or maintaining tight junctions and/or adherens junctions of the gut barrier.

Undesirable physiological changes to any part of the gut barrier, including the epithelial cells of the gut mucosa, may lead to an impaired or “leaky” gut barrier, and this may compromise the integrity of the barrier and result, e.g., in a “leaky gut syndrome” and a low-grade or chronic inflammation of the gut or other (extra-intestinal) bodily organs. The compositions according to the present invention are believed to be capable of treating a leaky gut syndrome in an individual, including any low-grade or chronic inflammation associated with a leaky gut syndrome.

A “leaky gut syndrome” is associated with an aberrant or impaired gut barrier function, including an aberrant or impaired gut barrier permeability, and one or more of chronic diarrhea, constipation, or bloating; nutritional deficiencies due to nutritional malabsorption; and various skin disorders, such as, e.g., acne, rashes, or eczema, as well as joint pain, including joint pain caused by arthritis.

Physiologically unhealthy increases in gut permeability and/or inflammation of the gut are conditions that are often associated with an imbalance or dysbiosis of the gut microbiota. The human gut eco-system consists of a variety of different habitats and metabolic niches, and the gut is colonized by a microbiota that contains more than 1011 microorganisms per gram (wet weight) gut tissue.

A physiologically balanced gut microbiota maintains health and well-being in an individual, e.g., by communicating and interacting with the immune system and promoting resistance to colonization of opportunistic pathogens. The gut microbiota is affected by numerous external perturbations, such as, e.g., dietary changes, use of antibiotics, exposure to pathogens, increasing hygienization, stress and fatigue. Each of these factors may lead to a condition in the microbiota, which is termed dysbiosis.

Dysbiosis is often characterized as moderate or severe changes to a physiologically balanced gut microbiota that generate an unwanted health-related outcome. The imbalance of the microbiota may lead to excessive production of undesirable metabolites that are harmful to and, e.g., disrupts the junctions between epithelial cells in the gut mucosa. The disruption of epithelial cell junctions will generate an increased para-cellular (i.e. between cells) permeability of the gut barrier. The weakening of gut epithelial cells may also generate an increased trans-permeability of the gut barrier, i.e. increased permeability through the epithelial cells.

Dysbiosis may also lead to a compromised immune response, nutrient malabsorption and low-grade and/or chronic inflammation in the gut and/or in extra-intestinal organs. The adverse effects of dysbiosis toward microbial functionality and endothelial cell physiology may thus undermine human health.

Dysbiosis caused by imbalances in the gut microbiota may lead to transient or longer lasting dysfunctions of the gut, gut barrier modulation or impairment, and chronic inflammatory disease states, such as, e.g. ulcerative colitis and Crohn's disease. Dysbiosis may also lead to colonization in the gut of pathogens capable of causing, e.g., diarrhea, including Clostridium difficile associated diarrhea, and/or necrotizing enteritis.

The compositions according to the present invention are believed to be capable of treating dysbiosis and dysbiosis associated diseases in an individual either by administration of the compositions alone or in combination with an antibiotic treatment.

According to presently preferred hypotheses, the compositions according to the present invention are capable of one or more of:
stimulating growth and/or activity of one or more beneficial microbial species in the gut,
inhibiting or reducing growth and/or metabolic activity of one or more non-beneficial or pathogenic microbial species in the gut,
facilitating attachment of non-pathogenic or beneficial microorganisms to the endothelial mucosa of the gut,
reducing uncontrolled transport or contact by endothelial cells of the gut of one or more of antigens, pro-inflammatory microorganisms, or undesirable microbial metabolites or products,
reducing unwanted or uncontrolled paracellular and/or transcellular influx from the gut and into the body of one or more of antigens, pro-inflammatory microorganisms or unwanted microbial metabolites or products,
facilitating uptake or metabolism by endothelial cells of the gut of diet nutrients and/or beneficial metabolites,
providing an anti-inflammatory activity at the epithelial cell surface of the gut,
improving gut barrier functioning and/or permeability, and
producing beneficial metabolites useful for a desirable gut barrier function and/or permeability.

In one aspect of the invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of impaired or aberrant gut barrier function in an individual, including a human being.

In another aspect of the invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of extra-intestinal low-grade or chronic inflammation associated with or caused by an impaired or aberrant gut barrier function in an individual, including a human being.

In yet another aspect of the invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of low-grade or chronic inflammation in the gut associated with or caused by an impaired or aberrant gut barrier function in an individual, including a human being.

In a still further aspect of the present invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of extra-intestinal low-grade or chronic inflammation associated with or caused by an impaired or aberrant gut barrier permeability in an individual, including a human being.

In yet another aspect of the present invention, there is provided a composition comprising at least 10 grams (dry weight) of fermented cereal obtained by fermenting a cereal gruel with one or both of Lactobacillus plantarum 299 and Lactobacillus plantarum 299 v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of low-grade or chronic inflammation in the gut associated with or caused by an impaired or aberrant gut barrier permeability in an individual, including a human being.

In a still further aspect of the present invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of impaired or aberrant gut barrier permeability in an individual, including a human being.

In another aspect of the invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299 v, or a related Lactobacillus cell, and fermented cereal obtained by fermenting a cereal gruel with at least one of said Lactobacillus cells, for use in a method of treatment of extra-intestinal low-grade or chronic inflammation associated with or caused by an impaired or aberrant gut barrier permeability in an individual, including a human being.

In yet another aspect of the invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299 v, or a related Lactobacillus cell, and fermented cereal obtained by fermenting a cereal gruel with at least one of said Lactobacillus cells, for use in a method of treatment of low-grade or chronic inflammation in the gut associated with or caused by an impaired or aberrant gut barrier permeability in an individual, including a human being.

In a still further aspect of the present invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus plantarum 299 cell, and fermented cereal obtained by fermenting a cereal gruel with at least one of said Lactobacillus plantarum 299 cells, for use in a method of treatment of extra-intestinal low-grade or chronic inflammation associated with or caused by an gut inflammation and an impaired or aberrant gut barrier permeability in an individual, including a human being.

In yet another aspect of the present invention, there is provided a composition comprising at least 10 grams (dry weight) of fermented cereal obtained by fermenting a cereal gruel with one or both of Lactobacillus plantarum 299 and Lactobacillus plantarum 299v cells, or a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells, for use in a method of treatment of low-grade or chronic inflammation in the gut as well as an extra-intestinal low-grade or chronic inflammation associated with or caused by inflammation of the gut and an impaired or aberrant gut barrier function and/or gut barrier permeability in an individual, including a human being.

There is also provided a method of treating impaired or aberrant gut barrier function and/or impaired or aberrant gut barrier permeability in an individual, including a human being, said method comprising the step of administering to said individual a composition comprising at least 10 grams (dry weight) of fermented cereal obtained by fermenting a cereal gruel with one or both of Lactobacillus plantarum 299 and Lactobacillus plantarum 299v cells, or administering a composition comprising at least one of Lactobacillus plantarum 299 and Lactobacillus plantarum 299v, or a related Lactobacillus cell, and fermented cereal obtained by fermenting a cereal gruel with at least one of said Lactobacillus cells.

In a still further aspect, there is provided a ready-to-use product comprising at least 10 grams (dry weight) of fermented cereal obtained by fermenting a cereal gruel with one or both of Lactobacillus plantarum 299 and Lactobacillus plantarum 299v cells, or a ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of aberrant or impaired gut barrier function in a human being. The aberrant or impaired gut barrier function is preferably associated with an aberrant or impaired gut barrier permeability-related disease or disorder, such as, e.g., Irritable Bowel Syndrome (IBS), ulcerative colitis or Crohn's disease.

In yet another aspect of the invention, there is provided a ready-to-use product comprising at least 10 grams (dry weight) of fermented cereal obtained by fermenting a cereal gruel with one or both of Lactobacillus plantarum 299 and Lactobacillus plantarum 299v cells, or a ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of inflammation of the gastrointestinal tract or an extra-intestinal organ in a human being. The inflammation is preferably associated with a gut permeability-related disease or disorder. Exemplary inflammations are, e.g., Irritable Bowel Syndrome (IBS), ulcerative colitis, Crohn's disease, extra-intestinal chronic inflammation, extra-intestinal low-grade inflammation, and extra-intestinal low-grade chronic inflammation.

In a still further aspect of the invention, there is provided a ready-to-use product comprising at least 10 grams (dry weight) of fermented cereal obtained by fermenting a cereal gruel with one or both of Lactobacillus plantarum 299 and Lactobacillus plantarum 299v cells, or a ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of dysbiosis of the gastrointestinal tract in a human being. The dysbiosis is preferably associated with a gut permeability-related disease or disorder, including Irritable Bowel Syndrome (IBS), ulcerative colitis and Crohn's disease.

The above-cited methods of treatment may be prophylactic, ameliorating or curative. A prophylactic treatment preferably comprises steps that maintain or protect gut barrier function in an individual, including a human being. An ameliorating treatment is capable of reducing at least one symptom of the clinical condition or disease being treated, whereas curative treatment is a treatment of essentially all symptoms of the clinical condition or disease.

Oatmeal is a preferred cereal gruel, and the fermentation of the cereal gruel, preferably oat meal, is preferably performed until the fermented gruel has a pH below about 5.5, such as below about 5.0, for example below 4.5, such as below about 4.0. The fermentation should be allowed to continue at 37° C. until a desirable pH value is obtain. Typical durations are from 12 to 24 hours, but shorter or longer fermentations may be performed as deemed preferred by the practitioner.

The compositions and ready-to-use products are preferably administered daily to an individual in need thereof, both variations to daily administrations may occur, as described under the section containing the definitions.

An individual is preferably a human being, but other mammals are also included in this definition.

If deemed desirable one or more Lactobacillus cells, such as Lactobacillus plantarum cells, for example Lactobacillus plantarum 299 or Lactobacillus plantarum 299v, may be added to the fermented cereal during or after the fermentation.

The compositions for use and ready-to-use products are drinkable or edible, and the compositions and ready-to-use products may be on a liquid form, a solid form, or a semi-solid form. The solid or semi-solid forms of the compositions and ready-to-use products may be obtained by drying the fermented cereal composition or removing water from the fermented cereal composition. It is also possible to subject the fermented cereal composition to a a heat treatment prior to obtaining a final composition or a final ready-to-use product.

The compositions and ready-to-use products according to the invention may contain desirable additives, including added lecithin and/or added phosphatidyl-choline.

The present invention is also directed to methods and compositions for use in preventing or treating diseases or clinical disorders associated with or caused by an impaired or aberrant gut barrier function and/or an impaired or aberrant gut barrier permeability in an individual.

The present invention also provides methods and compositions for use in preventing or treating impaired or aberrant gut permeability-related diseases and disorders in an individual.

Examples of diseases or clinical disorders associated with or caused by an impaired or aberrant gut barrier function and/or an impaired or aberrant gut barrier permeability in an individual, including impaired or aberrant gut barrier permeability-related diseases and disorders in the gut, are, e.g., gastric ulcers, infectious diarrhea, irritable bowel disease (IBS), inflammatory bowel disease (IBD), including Crohn's disease and ulcerative colitis, celiac disease and cancer, including cancer of the esophagus and colorectal cancer.

Examples of diseases or clinical disorders associated with or caused by an impaired or aberrant gut barrier function and/or an impaired or aberrant gut barrier permeability in an individual, including impaired or aberrant gut permeability-related extra-intestinal diseases and disorders (i.e. diseases or disorders in bodily organs other than the gut), are, e.g., allergies, auto-immune diseases, infections, inflammations, including low-grade inflammations (typically “low-grade” by e.g. defying a diagnosis), chronic inflammations, including arthritis, and acute inflammations, including sepsis, systemic inflammatory response syndrome (SIRS), multiple organ dysfunction syndrome (MODS) and multiple organ failure (MOF), as well as metabolic diseases, including metabolic syndrome associated with one or more of abdominal obesity, high blood pressure, impaired fasting glucose, high triglyceride levels, and low HDL cholesterol levels, as well as other types of obesity-associated metabolic diseases, including diabetes (of either type I of Type II), and cardiovascular diseases.

### Definitions

Aberrant: Deviating from a physiologically normal situation to such an extent that the deviation causes an unhealthy state of health, including one or more clinical symptoms of diagnostic significance, including, e.g., tissue inflammation, including low-grade and/or chronic inflammation. When relating to an aberrant gut barrier permeability, such a permeability is characterized by translocation over the gut barrier of substances which are not translocated in a physiologically normal gut barrier, including a healthy gut barrier with no inflammation and no signs of intoxication. A physiologically normal gut barrier permeability is defined as a stable permeability in healthy individuals with no signs of intoxication, inflammation, or impaired gut functions, whereas a disturbed gut permeability denotes a condition resulting in loss of gut homeostasis, functional gut impairments and gut permeability-related disease or disorder of the gut and/or other bodily organs.

Administration: An administration may be a daily administration, but other types of administration are also possible, including, e.g., twice daily, every second day, weekly and twice weekly administration.

Cereal: Any plant from the grass family that yields an edible grain (seed). The most common grains are barley, corn, millet, oats, quinoa, rice, rye, sorghum, triticale, wheat and rice. Oats are preferred.

Cereal gruel: Cereal grains, flakes, meal, extracts or flour boiled in a liquid, preferably is water. The cereal gruel is prepared in order to sterilize the suspended cereal, to convert some of the macromolecules in the cereal and to prepare the gruel for microbial fermentation. Enzymes, compositions containing enzymes and/or appropriate amounts of carbon and energy sources may be added to further prepare or facilitate the microbial fermentation and/or provide a desired rheology. Cereal gruel covers any rheological form of suspended and boiled cereal.

Chronic inflammation: Inflammation (cf. below) can be either short-lived (acute) or long-lasting (chronic). Acute inflammation typically goes away within hours or days. Chronic inflammation may last several weeks, months, or even years in severe cases. The following clinical conditions have been linked to chronic inflammation: Cancer, cardiovascular diseases, diabetes of type I or II, asthma and Alzheimer's disease. A chronic inflammation may be a low-grade inflammation.

Crohn's disease: Crohn's disease is an inflammatory bowel disease (IBD) characterized by chronic inflammation in the gastrointestinal (GI) tract. The clinical symptoms are diarrhea or constipation, abdominal pain, occasional rectal bleeding, weight loss, tiredness and sometimes fever.

Digestive Stool Analysis: An analysis of stool samples aimed at determining the presence of microbial organisms, including bacteria, yeast and fungi potentially associated with a clinical condition in an individual, including a human being, including aberrant gut permeability, inflammation and dysbiosis. Test may include both qualitative and quantitative results.

Dysbiosis: An imbalance of the gut microbiota in an individual associated with the appearance, metabolic activity or overgrowth of one or more of undesirable microorganisms, including pathogens, a decreased bacterial diversity, aberrant gut permeability and inflammation associated with an unwanted health-related outcome.

Fermented cereal composition: A composition resulting from the growth of one or both of Lactobacillus plantarum 299 (Lp299)/Lactobacillus plantarum 299v (Lp299v) or related lactobacilli in a cereal gruel. When oats are used as the cereal, the composition is described as a composition comprising fermented oat gruel.

Gut: Also known as the “gastrointestinal tract”. The gut includes the stomach, the small intestine (duodenum, jejunum, ileum) and the large intestine (cecum, colon). The duodenum mixes digestive juices from the pancreas and the liver, and the jejenum connects the duodenum to the ileum. Small molecules are absorbed into the blood through the villi of the ileum. The colon consists of the ascending colon, the transverse colon, the descending colon and the sigmoid flexure. The vermiform appendix is attached to the cecum. Tissues forming the mucosal surface of the gut are referred to as “gut tissue” or “mucosal tissue”.

Gut barrier: Functional barrier separating the gut lumen from inner bodily tissue and organs. The gut barrier comprises mucus and an epithelial layer, as well as humoral elements, including defensins and immununological elements, including IgA, lymphocytes and innate immune cells, as well as muscular and neurological elements.

Gut permeability: A functional feature of the permeability of the gut barrier at predetermined sites. The gut permeability may be measured by analyzing the flux (flow rate) of defined molecules across the gut barrier (intestinal wall) or parts thereof. The defined molecules in question should be essentially inert during the process so that they may be adequately measured during various assay settings. The permeability of the gut barrier of an individual is typically measured by using well known gut permeability assays and/or assessment of markers of epithelial integrity. Markers include, e.g., adhesion molecules, biomarkers of immunity or inflammation, as well as bacterial markers, as described, e.g., by Seethaler et al. (2021) in Am. J. Physiol. Gastrointest. Liver Physiol., vol. 321, G11-G17, and by Bischoff et al. (2014) in BC Gastroenterology, vol. 14, pp. 189 (pp. 1-25). A physiologically normal gut permeability is defined as a stable permeability in healthy individuals with no signs of intoxication, inflammation or impaired gut functions, whereas a disturbed gut permeability denotes a condition resulting in loss of gut homeostasis, functional gut impairments and gut permeability-related disease or disorder of the gut and/or other bodily organs.

Gut permeability-related disease or disorder: A disease or disorder associated with a disturbed, impaired or aberrant intestinal (gut) permeability. A disturbed, impaired or aberrant intestinal permeability is increased or decreased compared to a normal gut permeability. A disturbed, impaired or aberrant gut permeability typically leads to a reduction or loss of gut homeostasis, functional gut impairment and disease, including inflammation of the gut. A subject can be identified as suffering from disturbed or aberrant gut permeability as described, e.g., by Bischoff et al. (2014) (ibid.) and by Seethaler et al. (2021) (ibid.).

Gut microbiota: The entire content of gut microorganisms in an individual, such as a human being. Gut microorganisms may be commensal, symbiotic, or pathogenic.

Hydrogen Breath Test: A test for detecting gases produced by microbial organisms in the gut, including bacteria. In a hydrogen breath test, an individual drinks a glucose or sugar solution, and exhaled air is examined for the presence of gases produced by the gut microbiome. Aberrant amounts of gases indicate an imbalance in the gut microbiome, including dysbiosis.

Impaired: Deviating from a physiologically normal situation due to tissue damage or modification caused by, e.g., inflammation and/or intoxication (exposure to toxic substances). The deviation occurs to such an extent that an unhealthy state of health is generated, including an unhealthy state of health associated with one or more clinical symptoms of diagnostic significance, including, e.g., tissue inflammation, including low-grade and/or chronic inflammation. When relating to an aberrant gut barrier permeability, such a permeability is characterized by translocation over the gut barrier of substances which are not translocated in a physiologically normal gut barrier, including a healthy gut barrier with no inflammation and no signs of intoxication. A physiologically normal gut barrier permeability is defined as a stable permeability in healthy individuals with no signs of intoxication, inflammation or impaired gut functions, whereas a disturbed gut permeability denotes a condition resulting in loss of gut homeostasis, functional gut impairments and gut permeability-related disease or disorder, including inflammation, of the gut and/or other bodily organs.

Individual: A mammal, including a human being. In certain embodiments, the human being does not suffer from IBS and/or IBD.

Inflammation: A response, including an immunological response, of living tissues to undesirable physiological changes, including unhealthy changes that are associated with one or more clinical symptoms of diagnostic significance. The etiology of an inflammation may, e.g., be infectious, physical, chemical, vascular or dysimmunitary. An inflammation may be or develop into a chronic condition, including low-grade chronical inflammation in an individual.

Irritable Bowel Syndrome (IBS): Irritable Bowel Syndrome (IBS) is a gastrointestinal disorder with symptoms such as non-cardiac chest pain, non-ulcer dyspepsia, chronic constipation or diarrhea. IBS is characterized by chronic or recurrent gastrointestinal symptoms for which no structural or biochemical cause can be found.

Lactobacillus plantarum 299 (Lp299)/Lactobacillus plantarum 299v (Lp299v): Lactobacillus plantarum 299 has been deposited as DSM 6595, and Lactobacillus plantarum 299 v has been deposited as DSM 9843.

Low-grade inflammation: Low-grade inflammation is a chronic inflammatory response that generates a steady, low level of inflammation in selected bodily organs or throughout the entire body. In one embodiment, low-grade inflammation is defined by increased levels of C-reactive protein (CRP). CRP is one of the main inflammation biomarkers present in the body. The higher the CRP levels, the more inflammation is present throughout the body. Low-grade inflammation may be defined by CRP levels of from about 3 mg/L to about 10 mg/L. In low-grade inflammation, the body will typically continue to generate inflammatory cells, even when the cause of the inflammation has subsided. In rheumatoid arthritis, inflammatory cells and substances may continue to attack joint tissues, and this will lead to an inflammation in the joint tissues. This may cause severe damage to joints and cause both pain and deformities. A low-grade inflammation may develop into a chronic low-grade inflammation. A low-grade inflammation differs from normal inflammation in that there are no typical signs of inflammation, but it is similar in that it shares the disorders generated by typical inflammation mediators and signaling pathways.

Non-pathogenic microorganisms: Any microbial cell that is not harmful to humans or animals.

Organic Acids Test: Quantitative or qualitative test for organic acids that involves, e.g., collecting a body fluid sample, such as a urine sample, from an individual, such as a human being. The test allows a medical practitioner to determine, e.g., whether the gut microbiota of an individual produces physiologically normal or abnormal levels of organic acids. The medical practitioner may analyze the sample for the presence and/or amounts (incl.

concentration) of organic acids, and the medical practitioner is able to evaluate whether the presence and/or amount of at least one organic acid in the sample are indicative of a clinical indication in the individual being tested, including an aberrant gut permeability, inflammation and dysbiosis.

Ready-to-use product: A pre-packaged product which may be dispensed with only a minimal effort or preparation.

SIBO: Small intestinal bacterial overgrowth (SIBO) is a condition that occurs when there is an abnormal increase in the overall bacterial population in the small intestine.

Treatment: Treatment covers any form of treatment of a clinical condition or disease which is either prophylactic, ameliorating or curative. A treatment of a clinical indication an individual, such as a human being, includes both prophylaxis and ameliorating treatment resulting in prevention or amelioration of at least one symptom associated with the clinical indication in question.

Ulcerative colitis: Ulcerative colitis (UC) is an inflammatory bowel disease (IBD) characterized by chronic inflammation in the colon. The clinical symptoms are diarrhea, abdominal pain, occasional rectal bleeding, weight loss, tiredness and sometimes fever.

### BRIEF DISCLOSURE OF THE DRAWINGS

FIG. 1 illustrates the interrelationship in an individual between gut permeability, inflammation and dysbiosis.

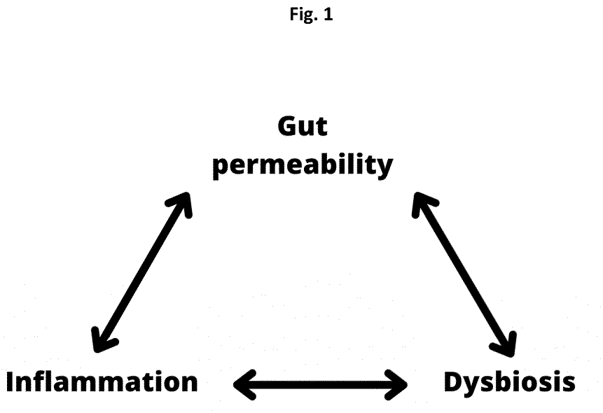

### DETAILED DESCRIPTION OF THE INVENTION

### Inflammation and Diagnosis Thereof

Inflammation is a highly conserved defensive mechanism capable of eliminating microorganisms and repairing tissue. It is characterized by the activation of immune and non-immune cells that provide surveillance and protection against a full spectrum of microorganisms and toxic insults.

Normal inflammatory responses are represented by acute and time-limited upregulation of the innate inflammatory response. Typically, this acute innate immune response is short and self-resolves once the threat has been eliminated. However, not all inflammatory responses follow this path, and chronic or “low-grade” inflammation may persist in an individual over prolonged periods of time. Chronic or “low-grade” inflammations may cause inflammation related diseases to develop, and chronic or “low-grade” inflammations are often associated with a variety of different types of diseases.

To diagnose inflammation in a human being, healthcare professionals may use various markers and tests, including, but not limited to:
C-reactive protein (CRP): CRP is a protein produced by the liver in response to inflammation. Elevated levels of CRP in the blood can indicate the presence of inflammation.
Erythrocyte sedimentation rate (ESR): ESR measures how quickly red blood cells settle in a tube of blood. Increased ESR values can be a sign of inflammation.
Complete blood count (CBC): This test provides information about the number of white blood cells, which are often elevated during inflammation.
Procalcitonin: Elevated levels of procalcitonin can indicate bacterial infection and inflammation.
Interleukins and cytokines: These are immune system molecules that play a key role in regulating inflammation. Elevated levels of certain interleukins or cytokines can suggest inflammation.
Imaging tests: X-rays, ultrasounds, CT scans, and MRIs can help visualize and identify inflamed tissues or organs.
Biopsy: In some cases, a small sample of tissue may be taken for examination under a microscope to confirm the presence of inflammation and identify its cause.

Specific markers used for diagnosing inflammation may vary depending on the underlying condition and the clinical judgment of the healthcare provider.

The molecular basis of inflammation in the human body involves a complex interplay of various cells, signaling molecules, and biochemical pathways. The immune system is activated during inflammation, and the activation of the immune system initiate the inflammatory response of the human body.

Some of the molecular processes involved in an inflammation are briefly reviewed below:
Recognition of Pathogens or Damage-Associated Molecules: The immune system's first step is to recognize the presence of pathogens or damage-associated molecules. This is achieved through pattern recognition receptors (PRRs) present on immune cells. PRRs can detect specific molecular patterns commonly found on pathogens or released by damaged cells.
Activation of Immune Cells: Upon recognition, immune cells, particularly macrophages and dendritic cells, are activated. These cells release pro-inflammatory cytokines (such as interleukin-1 (IL-1), interleukin-6 (IL-6), and tumor necrosis factor-alpha (TNF-alpha)) that act as signaling molecules, triggering and amplifying the inflammatory response.
Vasodilation and Increased Vascular Permeability: The pro-inflammatory cytokines cause the blood vessels near the affected area to dilate (vasodilation) and become more permeable. This allows an increased flow of blood to the site, leading to the characteristic redness and heat associated with inflammation. Increased vascular permeability allows immune cells, antibodies, and other molecules to move from the bloodstream into the tissues, contributing to swelling.
Recruitment of Immune Cells: Chemokines, a type of cytokine, are released, attracting various immune cells, including neutrophils and monocytes, to the site of inflammation. Neutrophils are the first responders, arriving early to combat infections, while monocytes mature into macrophages that play a more extended role in tissue repair and resolution of inflammation.
Phagocytosis and Destruction of Pathogens: Neutrophils and macrophages engulf and destroy pathogens through a process called phagocytosis. This helps eliminate the source of the inflammation.
Activation of the Complement System: The complement system, a group of proteins in the blood, is activated during inflammation. It enhances the immune response by assisting in pathogen destruction, attracting immune cells, and promoting inflammation.
Tissue Repair and Resolution: As the inflammatory process progresses, other immune cells, such as T lymphocytes, become involved in regulating the response and promoting tissue repair. Anti-inflammatory cytokines (such as interleukin-10 (IL-10)) are released, helping to control the inflammation and prevent excessive tissue damage. Once the threat is neutralized, the inflammatory response is normally resolved, and tissue repair processes take over. However, in some cases, an inflammatory response is not adequately resolved by the bodily defense systems, and this may turn an acute inflammation into a chronic inflammation.

An uncontrolled or chronic inflammatory response will often contribute to a variety of diseases in the body. Chronically inflamed tissues send out signaling compounds that will continue to trigger the immune response long after the initial threat has been dealt with by an acute inflammatory response.

For example, white blood cells may begin to attack healthy tissues and organs, and this action will likely amplify the inflammatory response and generate a longer lasting and potentially chronic inflammatory state.

### Treatment of Inflammation in Metabolic Diseases

The present invention provides in one aspect thereof compositions and ready-to-use products for use in treating inflammation in an individual suffering from a metabolic disease. The inflammation may be a chronic or low-grade inflammation.

The metabolic disease can, e.g., be associated with the metabolic syndrome and involve one or more of abdominal obesity, high blood pressure, impaired fasting glucose, high triglyceride levels, and low HDL cholesterol levels, as well as obesity-associated metabolic diseases, including diabetes (of either type I or Type II).

Chronic inflammation may induce a broad range of deficits in lipid metabolism, including decreases in serum HDL, and increases in triglycerides, lipoprotein a (Lp(a)) and low density lipoprotein (LDL).

The sustained levels of inflammation resulting from changes in lipid homeostasis is a contributor to the risk of contracting atherosclerosis. In addition to affecting serum lipid levels, chronic inflammation in metabolic diseases may adversely affect lipoprotein function. For example, the ability of high density lipoprotein (HDL) to prevent oxidation of LDL is severely diminished, and several steps in reverse cholesterol efflux are also affected by the continued activation of the immune system in a chronic inflammatory action in a metabolic disease.

One effect of a chronic inflammation in metabolic diseases is that inflamed bodily tissues will be subjected to long-term degradation during continued action by the inflammation defense systems. For example, when pro-inflammatory cells remain in the blood vessels, the pro-inflammatory cells promote formation of a substance called “sticky plaque”. This is the same plaque which is a contributing factor in many metabolic and cardiovascular diseases.

### Treatment of Inflammation in Liver Diseases

The compositions and ready-to-use products according to the present invention may also be used in the treatment of chronic or low-grade inflammation in various types of liver diseases.

In this aspect of the invention there is provided compositions and ready-to-use products for use in treating inflammation in an individual suffering from a liver disease. The inflammation may be a chronic high or low-grade inflammation.

The present invention makes it possible to treat inflammation in, e.g., fatty liver diseases and fibrotic liver diseases, by administering fermented cereal compositions and ready-to-use products comprising such compositions to an individual in need thereof.

According to presently preferred hypotheses, probiotic and/or postbiotic effects in the gut exerted by the claimed fermented cereal compositions prevent liver disease progression and liver disease development by treating the gut microbial dysbiosis and/or an impaired gut barrier leading to the inflammation associated with these diseases.

It may be possible to initiate at least a partial regression of a liver disease by treating the inflammation which is associated with the disease. Alternatively, it may be possible to postpone the progression of a liver disease by treating chronic or low-grade inflammation associated with the liver disease, including a fatty liver disease or a fibrotic liver disease.

The present invention also aims to treat gut dysbiosis with the aim of preventing liver disease progression and liver disease development, initiating regression of a liver disease, or postponing the progression of a liver disease, including a fatty liver disease or a fibrotic liver disease.

A fibrotic liver disease is characterized by the accumulation of fibrosis in the liver. Research indicates that gut microbial dysbiosis may play an important role in the pathogenesis in alcoholic liver disease (ALD), non-alcoholic liver disease (NAFLD), some types of cholestatic liver disease (CLD) and idiosyncratic drug induced liver disease (DILI).

Liver diseases are progressive and traditionally divided in following stages. The milder liver diseases include fat accumulation in the liver (steatosis), and the more harmful stages include inflammation (steatohepatitis) that often leads to fibrosis (scar tissue), cirrhosis (severe fibrosis and decreased liver function) and sometimes even liver cancer. By treating the cause of inflammation in a liver disease, the compositions and ready-to-use products are capable of at least slowing or eliminating the progression of the disease. Preferably, treatment of inflammation associated with the liver diseases described herein will initiate at least partial regression of the liver disease.

The following blood markers, assays, scores and imaging methods can be used for identifying and monitoring the progression of a liver disease: Bilirubin, albumin, coagulation factors, international normalized ratio, prothrombin time, mean corpuscular volume (MCV), aspartate aminotransferases (AST), alanine aminotransferases (ALT), alkaline phosphatases, immunoglobuline-A (IgA), gammaglutamyltranspeptidases (GGT), glutamic oxaloacetic transaminase (GOT), glutamic pyruvic transaminase (GPT), ferritin, platelet count, Enhanced Liver Fibrosis (ELF) test and elastography (Transient elastography and 2D Shear wave elastography).

Liver diseases can also be categorized based on etiology (the most likely cause of liver injury).

Exemplary liver diseases falling within the definition of a fatty liver or fibrotic liver disease used herein are introduced herein below.

Alcohol overuse is one of the main risk factors and etiological factors leading to liver disease. Alcoholic liver disease (ALD) is the umbrella term covering all stages of liver disease that is considered to be related to alcohol overuse. ALD includes alcoholic steatosis, alcoholic steatohepatitis, alcoholic liver fibrosis and alcoholic cirrhosis.

According to international guidelines the diagnosis of alcoholic liver disease (ALD) is based on a combination of features, including a history of significant alcohol intake (excess alcohol consumption>30 gram/day), clinical evidence of liver disease, and supporting laboratory abnormalities. Laboratory tests should be done to exclude other etiologies and to confirm the diagnosis.

Alcoholic fatty liver is an early and reversible consequence of excessive alcohol consumption. Fatty liver develops in the majority of individuals who consumes more than 60 g of alcohol per day, but may also occurs in people who drink less.

In some individuals alcohol consumption over a longer period leads to inflammation and alcoholic liver fibrosis. Appeal to alcohol reduction is currently the only effective intervention.

Alcoholic liver fibrosis (ALF) is the precursor of alcoholic cirrhosis. ALF and compensated cirrhosis rarely causes specific symptoms. It is suspected in individuals having a history of alcohol overuse and laboratory abnormalities suggesting liver ALD.

The diagnosis of alcoholic liver fibrosis and compensated cirrhosis are traditionally done by liver biopsy. The cut-off for significant ALF is Ishak Score 3-5 and Metavir F2-3, and the cut-off for cirrhosis is Ishak score 6 and Metavir F4. However, new non-invasive methods for diagnosing and grading liver fibrosis have been validated, but no consensus has been reached as to which methods to use in the clinic.

These measurements include imaging by shear wave and elastography and a panel of indirect serological makers used in algorithms to calculate the probability of liver fibrosis (Aspartate aminotransferases to platelet ratio index, FibroTest®, Fibrometer A®, Hepascore®).

The specific cut-off value has been correlated and therefore transmittable to fibrosis stages and cirrhosis due to the Ishak Score and Metavir. There is no treatment available for ALF and alcoholic cirrhosis except for appeal to alcohol reduction.

Alcoholic steatohepatitis (ASH) is a clinical syndrome, i.e. recent onset of jaundice and/or ascites in a patient with ongoing alcohol overuse or recently terminated alcohol overuse. Mild ASH is frequently observed in people with ALF without causing any symptoms, however severe ASH has a 90-days mortality of more than 30%.

Non-alcoholic fatty liver disease (NAFLD) is—like alcoholic fatty liver disease (ALD)—a generic term covering non-alcoholic fatty liver (NAFL), and non-alcoholic steatohepatitis (NASH) characterized by inflammation and fibrosis which may progress to cirrhosis, liver failure, and liver cancer.

NAFLD is associated with type 2 diabetes mellitus, dyslipidemia, obstructive sleep apnea, obesity and metabolic syndrome. The links between these diseases/disorders are not well understood.

To diagnose NAFLD it is required that (a) there is evidence of hepatic steatosis, either by imaging or by histology and (b) there are no causes for secondary hepatic fat accumulation such as significant alcohol consumption, use of steatogenic medication or hereditary disorders.

NAFLD is considered as an exclusion diagnosis in where other cause of liver injury should be excluded: Excessive alcohol consumption, hepatitis C (genotype 3), Wilson's disease, lipodystrophy, starvation, parenteral nutrition, abetalipoproteinemia, medications (e.g., amiodarone, methotrexate, tamoxifen, corticosteroids), microvesicular steatosis, Reye's syndrome, acute fatty liver of pregnancy, HELLP syndrome, inborn errors of metabolism.

Non-alcoholic fatty liver (NAFL) is regarded as a benign condition like obesity. Both of these conditions have a close relationship and predispose to cardiovascular diseases and diabetes mellitus. NAFL is regarded as a precursor to non-alcoholic steatohepatitis.

The presence of metabolic syndrome is a strong predictor for the presence of steatohepatitis in patients with NAFLD and may be used to best identify patients with persistently abnormal liver biochemistries.

Non-alcoholic steatohepatitis (NASH) is like alcoholic liver fibrosis traditionally diagnosed by liver biopsy, however non-invasive methods to identify advanced fibrosis in patients with NAFLD include the NAFLD Fibrosis Score, Enhanced Liver Fibrosis (ELF®) panel and transient elastography. There is no broad consensus on treatment of NASH, but the American Association of Studying the Liver recommends Pioglitazone and Vitamin E (a-tocopherol) to treat steatohepatitis in patients with biopsy-proven NASH.

The present invention is also directed to compositions and ready-to-use products for use in treating the cause(s) of inflammations, including chronic high and low-grade inflammations, in one or more of the above-cited liver diseases.

### Cereal Gruel Fermentation and Compositions Comprising Fermented Cereal Gruel

The fermented cereal gruel compositions according to the present invention are for use in preventing or treating diseases or clinical disorders related to an impaired or aberrant gut barrier function in an individual, or for use in preventing or treating impaired or aberrant gut permeability-related diseases and disorders in an individual. Exemplary diseases and disorders are listed herein elsewhere.

The compositions are obtained by fermenting a cereal gruel with a Lactobacillus cell, preferably Lactobacillus plantarum 299 and/or Lactobacillus plantarum 299v, or a related Lactobacillus cell. The fermentation may be performed with a Lactobacillus cell related to Lactobacillus plantarum 299 and/or Lactobacillus plantarum 299v and optionally one or more additional lactic acid bacterial cells.

The compositions according to the invention are made in two steps. In a first step, a cereal gruel is prepared. The cereal gruel may be made by mixing cereal and water. When the cereal is oat, e.g., 18.5% (w/w) oat meal and from about 0.9% to about 2.5% (w/w) malted barley flour is mixed with water. The mixture is slowly stirred and heated for about 10 to 20 minutes at 37° C. The temperature is then raised to about 90° C. to 100° C. for about 15 to 30 minutes. The resulting oat gruel is then cooled to a temperature of about 37° C.

The fermentation is performed in a second step. A starter culture comprising, e.g., Lactobacillus plantarum 299 or Lactobacillus plantarum 299v is added to the cereal gruel to initiate the fermentation. The amount of added starter culture having about 10⁹ coluny forming units (cfu) per ml may be, e.g., 0.01% (v/v), 0.1% (v/v) or 1.0% (v/v).

The fermentation is preferably carried out with mild stirring at about 37° C. for a period of typically from about 12 hours to 24 hours. The resulting fermentation product typically has a cfu number of about 109/ml. The pH of the fermentation product is preferably below about 5.0, such as below about 4.5, for example below about 4.0.

The fermentation product may be cooled to about 4° C. and packed in sterile, ready-to-use containers of a suitable volume, such as, for example, 250 ml. Alternatively, the fermentation product is heat treated or dried prior to packaging. Heat treatment and drying are processes aimed at prolonging the shelf life of the fermentation product and ready-to-use compositions according to the present invention.

Heat treating and/or drying the fermentation product may reduce the number of Lactobacillus cells capable of forming colony units (i.e. the cfu number). According to a presently preferred hypothesis, heat treatment and/or drying should be performed under as gentle as possible conditions so that the integrity of the Lactobacillus cells are maintained. Surprisingly, the number of viable Lactobacillus cells in the product after completion of the fermentation at 37° C. does not seem to be critical for obtaining a technical effect. It is presently believed that the number of Lactobacillus cells capable of forming colonies (i.e. the cfu number) is not critical once the fermentation at 37° C. has been completed.

In one embodiment of the present invention, the fermentation product is a post-biotic, ready-to-use product comprising cereal gruel fermented by Lactobacillus plantarum 299 or Lactobacillus plantarum 299v, or a related Lactobacillus cell.

The number of viable cells capable of forming colonies may be essentially zero, and preferably less than about 1000 cells, such as less than about 100 cells per gram (w/w) fermented cereal or ready-to-use product.

If deemed necessary, the fermentation product may be stored at 4° C., and the product will typically have a shelf life of at least two months, preferably at least four to six months, such as, e.g., from 12 to 24 months.

The compositions and ready-to-use products according to the present invention may be administered to an individual in need thereof on a daily basis. The compositions and ready-to-use products according to the invention may also be administered once a week or several times during a week, such as, e.g. twice weekly, or three times weekly, such as every second day.

The dosage regimen will typically depend on the dosage amount, the individual to whom the compositions or ready-to-use products are administered, and the diseases or clinical indications (if any) to be treated.

Typically, a daily dosage of at least 10 grams (dry weight) fermented cereal is administered on a daily basis, corresponding to a weekly dosage of at least 70 grams (dry weight) fermented cereal.

A daily dosage may be split into two or more daily sub-dosages, and in some cases, two or more daily dosages may be administered on the same day or on each day over a predetermined period of time, such as a week, a month, a year, or longer, in case some individuals are in need thereof.

Daily dosages higher than 10 grams (dry weight) fermented cereal may be administered to individuals in need thereof. Higher daily dosages may contain, e.g., at least 12 grams (dry weight) fermented cereal, such as at least 14 grams (dry weight) fermented cereal, for example at least 16 grams (dry weight) fermented cereal, such as at least 18 grams (dry weight) fermented cereal, for example at least 20 grams (dry weight) fermented cereal, such as daily dosages containing at least 22 grams (dry weight) fermented cereal, for example at least 24 grams (dry weight) fermented cereal, such as at least 26 grams (dry weight) fermented cereal, for example at least 28 grams (dry weight) fermented cereal, such as daily dosages containing at least 30 grams (dry weight) fermented cereal, for example at least 32 grams (dry weight) fermented cereal, such as at least 34 grams (dry weight) fermented cereal, for example at least 36 grams (dry weight) fermented cereal, such as at least 38 grams (dry weight) fermented cereal, for example daily dosages containing at least 40 grams (dry weight) fermented cereal, such as at least 42 grams (dry weight) fermented cereal, for example at least 44 grams (dry weight) fermented cereal, such as at least 46 grams (dry weight) fermented cereal, for example at least 48 grams (dry weight) fermented cereal, such as daily dosages containing at least 50 grams (dry weight) fermented cereal, for example at least 52 grams (dry weight) fermented cereal, such as at least 54 grams (dry weight) fermented cereal, for example at least 56 grams (dry weight) fermented cereal, such as at least 58 grams (dry weight) fermented cereal, for example daily dosages containing at least 60 grams (dry weight) fermented cereal, such as at least 62 grams (dry weight) fermented cereal, for example at least 64 grams (dry weight) fermented cereal, such as at least 66 grams (dry weight) fermented cereal, for example at least 68 grams (dry weight) fermented cereal, such as daily dosages containing at least 70 grams (dry weight) fermented cereal, for example at least 72 grams (dry weight) fermented cereal, such as at least 74 grams (dry weight) fermented cereal, for example at least 76 grams (dry weight) fermented cereal, such as at least 78 grams (dry weight) fermented cereal, for example daily dosages containing at least 80 grams (dry weight) fermented cereal, such as at least 82 grams (dry weight) fermented cereal, for example at least 84 grams (dry weight) fermented cereal, such as at least 86 grams (dry weight) fermented cereal, for example at least 88 grams (dry weight) fermented cereal, such as daily dosages containing at least 90 grams (dry weight) fermented cereal, for example at least 92 grams (dry weight) fermented cereal, such as at least 94 grams (dry weight) fermented cereal, for example at least 96 grams (dry weight) fermented cereal, such as at least 98 grams (dry weight) fermented cereal, for example daily dosages containing at least 100 grams (dry weight) fermented cereal, such as at least 105 grams (dry weight) fermented cereal, for example at least 110 grams (dry weight) fermented cereal, such as at least 125 grams (dry weight) fermented cereal, for example a daily dosage of at least 150 grams (dry weight) fermented cereal.

Impaired or aberrant gut permeability-related gut diseases include, e.g., gastric ulcers, infectious diarrhea, irritable bowel disease (IBS), inflammatory bowel disease (IBD), including Crohn's disease and ulcerative colitis, celiac disease and cancer, including cancer of the esophagus and colorectal cancer.

Impaired or aberrant gut permeability-related diseases or disorders in bodily organs other than the gut (i.e. extra-intestinal diseases) include, e.g., allergies, auto-immune diseases, infections, inflammations, including low-grade inflammations (typically “low-grade” by e.g. defying a diagnosis), chronic inflammations, including arthritis, and acute inflammations, including sepsis, systemic inflammatory response syndrome (SIRS), multiple organ dysfunction syndrome (MODS) and multiple organ failure (MOF), as well as metabolic diseases, including metabolic syndrome associated with one or more of abdominal obesity, high blood pressure, impaired fasting glucose, high triglyceride levels, and low HDL cholesterol levels, as well as other types of obesity-associated metabolic diseases, including diabetes (of either type I of Type II), and cardiovascular diseases.

When the aberrant or impaired gut barrier permeability is associated with inflammation of the intestinal cell wall, including inflammation of mucosal and submucosal tissues, the claimed compositions may be used for treating intestinal cell wall inflammation, including inflammation of mucosal and submucosal tissues thereof, including acute, low-grade or chronic inflammation. The treatment alleviates or eliminates any impaired or aberrant permeability of the gut barrier associated with the afore-mentioned diseases.

Other examples of impaired or aberrant gut permeability-related diseases or disorders include, e.g., irritable bowel syndrome (IBS), inflammatory bowel disease (IBD), including Crohn's disease and ulcerative colitis, as well as, e.g., type I diabetes, type II diabetes, obesity, rheumatoid arthritis and multiple sclerosis.

By restoring or protecting the gut barrier and/or the function of the gut barrier, including gut barrier permeability, the compositions and ready-to-use products according to the present invention improves or maintains a physiologically healthy level of digestion, absorption and gut barrier translocation of nutrients without, or essentially without, a concomitant translocation of harmful substances that cause, e.g., low-grade and/or chronic inflammation, including low-grade chronic inflammation, either systemically or in one or more extra-intestinal organs of the body of an individual, including a human being.

### Methods of Determining Gut Barrier Function and Permeability

The permeability of the gut barrier may be determined as described, e.g., by Bischoff (ibid.) and Seethaler (ibid.). Techniques used for determining permeability and integrity of the gut barrier may vary depending on the setting (in vitro versus in vivo measurements), the species (human or animal models), the marker molecules used for assessment (ions, carbohydrates of different sizes, macromolecules and antigens, bacterial products and bacteria), and the com-partments used for measurement of the marker molecules (peripheral blood, portal vein blood, urine).

The flux of molecules across the gut barrier depends on the type of molecules and the type of gut barrier function defects, if any. To measure gut barrier dysfunctions, an Ussing chamber may be used. This ex vivo approach to measure gut barrier permeability requires intestinal tissue specimens, either in the form of biopsies or surgical specimens.

In vivo assessment of gut barrier function and permeability in humans is possible by using any one of a number of different gut barrier permeability assays, and by assessment of biomarkers of epithelial integrity such as soluble adhesion molecules, other biomarkers of immunity or inflammation, or bacterial markers like circulating endotoxin (cf. Tables 4 and 5 in Bischoff et al. (2014)).

In addition, histological approaches and scanning electron microscopy analyses have been used in experimental settings. The most relevant methods for assessment of gut barrier function and permeability in clinical settings are described in more detail herein below.

An Ussing chamber allows the measurement of short-circuit current as an indicator of active ion transport taking place across the gut barrier. The Ussing chamber consists of two halves that are mounted together so that the apical side is isolated from the basolateral side of the gut tissue sample to be analysed. An active ion transport across the gut barrier produces a potential difference across the epithelium (VEP) which is indicative of the transport in question. Gut barrier permeability techniques using the Ussing chamber are well established and have been used successfully for many years.

In addition to the Ussing chamber methods, a series of permeability assays have been developed for determining gut barrier translocation of selected molecules, as also described by, e.g., Bischoff et al. (2014) and Seethaler et al. (2021), cf. above.

Permeability assays usually use probe molecules, such as, e.g., PEGs and various types of saccharides, such as, e.g., lactulose, or PEGs ranging in MW of from about 400 kDa to about 4000 kDa, as well as smaller molecules, such as, e.g., mono-and oligosaccharides, such as, e.g., mannitol, L-rhamnose, or other types of probes, such as, e.g., 51Cr-EDTA.

Larger sized molecules are believed to translocate the gut barrier only, if the gut barrier function is impaired. In case of a compromise or complete loss of gut barrier function, the larger sized molecules will cross the gut barrier by diffusion, or otherwise, and enter the circulation. The molecules can subsequently be detected in urine after renal excretion.

Smaller sized molecule are generally able to traverse the intestinal barrier more freely, and often irrespective of whether the gut barrier function has been compromised or lost. The ratio of the urinary concentration of both types of “probe” molecules may be measured after five to six hours. The ratio would depend, e.g., on the size/molecular weight of the “probe” molecues, the “mode” of the absorption or translocation (i.e. passive or active transport over the gut barrier), the site and rate of absorption, as well as the kinetics of distribution into different body compartments.

An alternative to the above-captioned “active” assessment of the function and permeability of the gut barrier would be a “passive” assessment of the functionality of the gut barrier. A “passive” assessment would be possible by quantification of, e.g., luminal compounds, such as, e.g., endotoxins and/or bacterial fermentation in the gut plasma as markers for gut barrier function and integrity.

### Items Relating to the Present Invention

1. A ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of aberrant or impaired gut barrier function in a human being.
2. The ready-to-use product according to item 1, wherein the aberrant or impaired gut barrier function is associated with an aberrant or impaired gut barrier permeability-related disease or disorder.
3. The ready-to-use product according to item 1, wherein the impaired gut barrier function is associated with Irritable Bowel Syndrome (IBS).
4. The ready-to-use product according to item 1, wherein the impaired gut barrier function is associated with ulcerative colitis.
5. The ready-to-use product according to item 1, wherein the impaired gut barrier function is associated with Crohn's disease.
6. The ready-to-use product according to any of items 1 to 5, wherein the method of treatment is prophylactic, ameliorating or curative.
7. The ready-to-use product according to any of items 1 to 6, wherein the cereal is oatmeal.
8. The ready-to-use product according to any of items 1 to 7, wherein the cereal gruel is fermented until the fermented cereal has a pH below 5.5.
9. The ready-to-use product according to any of items 1 to 8, wherein the use includes daily administration of the product to a human being.
10. The ready-to-use product according to any of items 1 to 9, wherein one or more Lactobacillus plantarum cells are added to the fermented cereal.
11. The ready-to-use product according to any of items 1 to 10, wherein the product is a drinkable or edible product.
12. The ready-to-use product according to any of items 1 to 11, wherein the product is on a liquid form.
13. The ready-to-use product according to any of items 1 to 11, wherein the product is on a solid or semi-solid form.
14. The ready-to-use product according to item 13, wherein the solid or semi-solid form is obtained by drying the fermented cereal or removing water from the fermented cereal.
15. The ready-to-use product according to any of items 1 to 14, wherein the fermented cereal is subjected to a heat treatment.
16. The ready-to-use product according to any of items 1 to 15, wherein the product comprises added lecithin or added phosphatidyl-choline.
17. A ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of inflammation of the gastrointestinal tract in a human being.
18. The ready-to-use product according to item 17, wherein the inflammation is associated with a gut permeability-related disease or disorder.
19. The ready-to-use product according to item 17, wherein the inflammation is associated with Irritable Bowel Syndrome (IBS).
20. The ready-to-use product according to item 17, wherein the inflammation is associated with ulcerative colitis.
21. The ready-to-use product according to item 17, wherein the inflammation is associated with Crohn's disease.
22. The ready-to-use product according to any of items 17 to 21, wherein the method of treatment is prophylactic, ameliorating or curative.
23. The ready-to-use product according to any of items 17 to 22, wherein the cereal is oatmeal.
24. The ready-to-use product according to any of items 17 to 23, wherein the cereal gruel is fermented until the fermented cereal has a pH below 5.5.
25. The ready-to-use product according to any of items 17 to 24, wherein the use includes daily administration of the product to a human being.
26. The ready-to-use product according to any of items 17 to 25, wherein one or more Lactobacillus plantarum cells are added to the fermented cereal.
27. The ready-to-use product according to any of items 17 to 26, wherein the product is a drinkable or edible product.
28. The ready-to-use product according to any of items 17 to 27, wherein the product is on a liquid form.
29. The ready-to-use product according to any of items 17 to 27, wherein the product is on a solid or semi-solid form.
30. The ready-to-use product according to item 29, wherein the solid or semi-solid form is obtained by drying the fermented cereal or removing water from the fermented cereal.
31. The ready-to-use product according to any of items 17 to 30, wherein the fermented cereal is subjected to a heat treatment.
32. The ready-to-use product according to any of items 17 to 31, wherein the product comprises added lecithin or added phosphatidyl-choline.
33. A ready-to-use product comprising one or both of Lactobacillus plantarum 299 and Lactobacillus plantarum 299v and at least 10 grams (dry weight) of fermented cereal obtained by fermenting a cereal gruel with one or both of said Lactobacillus plantarum cells, for use in a method of treatment of dysbiosis of the gastrointestinal tract in a human being.
34. The ready-to-use product according to item 33, wherein the dysbiosis is associated with a gut permeability-related disease or disorder.
35. The ready-to-use product according to item 33, wherein the dysbiosis is associated with Irritable Bowel Syndrome (IBS).
36. The ready-to-use product according to item 33, wherein the dysbiosis is associated with ulcerative colitis.
37. The ready-to-use product according to item 33, wherein the dysbiosis is associated with Crohn's disease.
38. The ready-to-use product according to any of items 33 to 37, wherein the method of treatment is prophylactic, ameliorating or curative.
39. The ready-to-use product according to any of items 33 to 38, wherein the cereal is oatmeal.
40. The ready-to-use product according to any of items 33 to 39, wherein the cereal gruel is fermented until the fermented cereal has a pH below 5.5.
41. The ready-to-use product according to any of items 33 to 40, wherein the use includes daily administration of the product to a human being.
42. The ready-to-use product according to any of items 33 to 41, wherein one or more Lactobacillus plantarum cells are added to the fermented cereal.
43. The ready-to-use product according to any of items 33 to 42, wherein the product is a drinkable or edible product.
44. The ready-to-use product according to any of items 33 to 42, wherein the product is on a liquid form.
45. The ready-to-use product according to any of items 33 to 42, wherein the product is on a solid or semi-solid form.
46. The ready-to-use product according to item 45, wherein the solid or semi-solid form is obtained by drying the fermented cereal or removing water from the fermented cereal.
47. The ready-to-use product according to any of items 33 to 46, wherein the fermented cereal is subjected to a heat treatment.
48. The ready-to-use product according to any of items 33 to 47, wherein the product comprises added lecithin or added phosphatidyl-choline.

### EXAMPLE 1

### Manufacture of a Ready-To-Use Product Consisting of Fermented Oat Gruel with Lactobacillus plantarum

The manufacture of a ready-to-use product consisting of fermented oat gruel with Lactobacillus plantarum cells is divided into two steps according to this recipe.

This first step covers the preparation of oat gruel. This step may be performed essentially as disclosed in EP 0 415 941 B1.

An alternative method is to mix 18.5% (w/w) oatmeal and 0.9-2.5% (w/w) malted barley flour with water. The mixture is slowly stirred and heated for 10-20 minutes at 37° C. and following 15-30 minutes at 90-100° C. The resulting oat gruel is cooled to a temperature of about 37° C. and is now ready for the second step namely the fermentation process. A starter culture consisting of Lactobacillus plantarum 299 or strain 299v is added to the oat gruel to initiate the fermentation.

The amount of added starter culture with a cfu number of about 10¹⁰/ml is 0.01, 0.1 or 1.0% (v/v). The fermentation is carried out with mild stirring at 37° C. for 12-24 hrs.

The resulting ready to use product with a cfu number of at least 108/ml, and a pH below 5.5, preferably a pH below 5.0,is then cooled at 4° C. and packed for instance in sterile storage containers of 250 ml, which have a shelf life of at least 12 months, and preferably a shelf life of at least about five months, when kept at 4° C.

### EXAMPLE 2

### Manufacture of a Ready-to-Use Product Consisting of Fermented Oat Gruel with Probiotic Microorganisms and Lecithin

A ready to use product consisting of fermented oat gruel with probiotic microorganisms is manufactured as described in Example 1.

Subsequently, lecithin is added. 12 grams of granulated lecithin, such as e.g. “Lecithin Granulat” from Biosym A/S, DK-7430 Ikast, Denmark, are added per liter fermented oat gruel with probiotic microorganisms.

The mixture is stirred for about one minute and kept overnight at 4° C. resulting in a ready-to-use product comprising fermented oat gruel and probiotic microorganisms and lecithin, wherein the product has a cfu number of at least about 10⁸/ml and pH below 5.5, preferably below 5.0. This product is packed for instance in sterile storage containers of 250 ml, which have a shelf life of at least two months, and preferably a shelf life of at least about 12 months, when kept at 4° C.

Larger or smaller amounts of lecithin, or other physical forms and qualities of lecithin, can be used in this protocol. Numerous commercial lecithins are available and most have different contents of phosphatidylcholine, lysophosphatidylcholine, phosphatidylinositol and phosphatidylethanolamine.

The fatty acids residues of these phospholipids may be saturated, mono-unsaturated or poly-unsaturated. Also, the lecithin to be used in this protocol may have different physical forms such as liquid, granulated, encapsulated or mixed with any other substances such as vegetable oils. Encapsulated lecithin include formulations where lecithin is designed to be released at specific locations in the gastrointestinal tract for instance as defined as “retarded release” by Stremmel et al. 2005 Gut 54:966-971.

1. A ready-to-use product comprising at least 10 grams (dry weight) of fermented cereal obtained by fermenting a cereal gruel with one or both of Lactobacillus plantarum 299 and Lactobacillus plantarum 299v cells, for use in a method of treatment of one or more of an inflammation of the gastrointestinal tract, a systemic inflammation, an extra-intestinal inflammation, including an inflammation of an extra-intestinal organ, in a human being.

2. The ready-to-use product according to claim 1, wherein one or both of said Lactobacillus plantarum 299 and Lactobacillus plantarum 299v cells are present in the ready-to-use product after the fermentation as live cells, attenuated cells, or inactivated or non-viable cells.

3. The ready-to-use product according to any of claims 1 and 2, wherein the inflammation is one or more of extra-intestinal chronic inflammation, extra-intestinal low-grade inflammation, and extra-intestinal low-grade chronic inflammation.

4. The ready-to-use product according to any of the preceding claims, wherein the inflammation is an extra-intestinal chronic inflammation.

5. The ready-to-use product according to claim 4, wherein the extra-intestinal chronic inflammation is associated with a disease in the human being.

6. The ready-to-use product according to claim 5, wherein the disease is one or more diseases selected from the group consisting of irritable bowel syndrome (IBS); inflammatory bowel disease (IBD), including Crohn's disease and ulcerative colitis, small intestine bacterial overgrowth (SIBO), a metabolic disease, including metabolic syndrome associated with one or more of abdominal obesity, high blood pressure, impaired fasting glucose, high triglyceride levels, and low HDL cholesterol levels, as well as diabetes of type I or Il.

7. The ready-to-use product according to claim 5, wherein the disease is a liver disease.

8. The ready-to-use product according to claim 7, wherein the liver disease is selected from an alcoholic liver disease (ALD) and a non-alcoholic fatty liver disease (NAFLD).

9. The ready-to-use product according to claim 7, wherein the liver disease is selected from cirrhosis and chronic hepatitis.

10. The ready-to-use product according to any of claims 1 to 9, wherein the method of treatment is prophylactic, ameliorating or curative.

11. The ready-to-use product according to any of claims 1 to 10, wherein the cereal is oatmeal.

12. The ready-to-use product according to any of claims 1 to 11, wherein the cereal gruel is fermented until the fermented cereal has a pH below 5.5.

13. The ready-to-use product according to any of claims 1 to 12, wherein the use includes daily administration of the product to a human being.

14. The ready-to-use product according to any of claims 1 to 13, wherein one or more Lactobacillus plantarum cells are added to the fermented cereal.

15. The ready-to-use product according to any of claims 1 to 14, wherein the product is a drinkable or edible product.

16. The ready-to-use product according to any of claims 1 to 14, wherein the product is on a liquid form.

17. The ready-to-use product according to any of claims 1 to 14, wherein the product is on a solid or semi-solid form.

18. The ready-to-use product according to claim 17, wherein the solid or semi-solid form is obtained by drying the fermented cereal or removing water from the fermented cereal.

19. The ready-to-use product according to any of claims 1 to 18, wherein the fermented cereal is subjected to a heat treatment.

20. The ready-to-use product according to any of claims 1 to 19, wherein the product comprises added lecithin or added phosphatidyl-choline.

21. A ready-to-use product comprising at least 10 grams (dry weight) of fermented cereal obtained by fermenting a cereal gruel with one or both of Lactobacillus plantarum 299 and Lactobacillus plantarum 299v cells, for use in a method of treatment of aberrant or impaired gut barrier function in a human being.

22. The ready-to-use product according to claim 21, wherein the aberrant or impaired gut barrier function is associated with an aberrant or impaired gut barrier permeability-related disease or disorder.

23. The ready-to-use product according to claim 21, wherein the impaired gut barrier function is associated with Irritable Bowel Syndrome (IBS).

24. The ready-to-use product according to claim 21, wherein the impaired gut barrier function is associated with ulcerative colitis.

25. The ready-to-use product according to claim 21, wherein the impaired gut barrier function is associated with Crohn's disease.

26. The ready-to-use product according to any of claims 21 to 25, wherein the method of treatment is prophylactic, ameliorating or curative.

27. The ready-to-use product according to any of claims 21 to 26, wherein the cereal is oatmeal.

28. The ready-to-use product according to any of claims 21 to 27, wherein the cereal gruel is fermented until the fermented cereal has a pH below 5.5.

29. The ready-to-use product according to any of claims 21 to 28, wherein the use includes daily administration of the product to a human being.

30. The ready-to-use product according to any of claims 21 to 29, wherein one or more Lactobacillus plantarum cells are added to the fermented cereal.

31. The ready-to-use product according to any of claims 21 to 30, wherein the product is a drinkable or edible product.

32. The ready-to-use product according to any of claims 21 to 31, wherein the product is on a liquid form.

33. The ready-to-use product according to any of claims 21 to 31, wherein the product is on a solid or semi-solid form.

34. The ready-to-use product according to claim 33, wherein the solid or semi-solid form is obtained by drying the fermented cereal or removing water from the fermented cereal.

35. The ready-to-use product according to any of claims 21 to 34, wherein the fermented cereal is subjected to a heat treatment.

36. The ready-to-use product according to any of claims 21 to 35, wherein the product comprises added lecithin or added phosphatidyl-choline.

37. A ready-to-use product comprising at least 10 grams (dry weight) of fermented cereal obtained by fermenting a cereal gruel with one or both of Lactobacillus plantarum 299 and Lactobacillus plantarum 299v cells, for use in a method of treatment of inflammation of the gastrointestinal tract in a human being.

38. The ready-to-use product according to claim 37, wherein the inflammation is associated with a gut permeability-related disease or disorder.

39. The ready-to-use product according to claim 37, wherein the inflammation is associated with Irritable Bowel Syndrome (IBS).

40. The ready-to-use product according to claim 37, wherein the inflammation is associated with ulcerative colitis.

41. The ready-to-use product according to claim 37, wherein the inflammation is associated with Crohn's disease.

42. The ready-to-use product according to any of claims 37 to 41, wherein the method of treatment is prophylactic, ameliorating or curative.

43. The ready-to-use product according to any of claims 37 to 42, wherein the cereal is oatmeal.

44. The ready-to-use product according to any of claims 37 to 43, wherein the cereal gruel is fermented until the fermented cereal has a pH below 5.5.

45. The ready-to-use product according to any of claims 37 to 44, wherein the use includes daily administration of the product to a human being.

46. The ready-to-use product according to any of claims 37 to 45, wherein one or more Lactobacillus plantarum cells are added to the fermented cereal.

47. The ready-to-use product according to any of claims 37 to 46, wherein the product is a drinkable or edible product.

48. The ready-to-use product according to any of claims 37 to 47, wherein the product is on a liquid form.

49. The ready-to-use product according to any of claims 37 to 47, wherein the product is on a solid or semi-solid form.

50. The ready-to-use product according to claim 49, wherein the solid or semi-solid form is obtained by drying the fermented cereal or removing water from the fermented cereal.

51. The ready-to-use product according to any of claims 37 to 50, wherein the fermented cereal is subjected to a heat treatment.

52. The ready-to-use product according to any of claims 37 to 51, wherein the product comprises added lecithin or added phosphatidyl-choline.

53. A ready-to-use product comprising at least 10 grams (dry weight) of fermented cereal obtained by fermenting a cereal gruel with one or both of Lactobacillus plantarum 299 and Lactobacillus plantarum 299v cells, for use in a method of treatment of dysbiosis of the gastrointestinal tract in a human being.

54. The ready-to-use product according to claim 53, wherein the dysbiosis is associated with a gut permeability-related disease or disorder.

55. The ready-to-use product according to claim 53, wherein the dysbiosis is associated with Irritable Bowel Syndrome (IBS).

56. The ready-to-use product according to claim 53, wherein the dysbiosis is associated with ulcerative colitis.

57. The ready-to-use product according to claim 53, wherein the dysbiosis is associated with Crohn's disease.

58. The ready-to-use product according to any of claims 53 to 57, wherein the method of treatment is prophylactic, ameliorating or curative.

59. The ready-to-use product according to any of claims 53 to 58, wherein the cereal is oatmeal.

60. The ready-to-use product according to any of claims 53 to 59, wherein the cereal gruel is fermented until the fermented cereal has a pH below 5.5.

61. The ready-to-use product according to any of claims 53 to 60, wherein the use includes daily administration of the product to a human being.

62. The ready-to-use product according to any of claims 53 to 61, wherein one or more Lactobacillus plantarum cells are added to the fermented cereal.

63. The ready-to-use product according to any of claims 53 to 62, wherein the product is a drinkable or edible product.

64. The ready-to-use product according to any of claims 53 to 62, wherein the product is on a liquid form.

65. The ready-to-use product according to any of claims 53 to 62, wherein the product is on a solid or semi-solid form.

66. The ready-to-use product according to claim 65, wherein the solid or semi-solid form is obtained by drying the fermented cereal or removing water from the fermented cereal.

67. The ready-to-use product according to any of claims 53 to 66, wherein the fermented cereal is subjected to a heat treatment.

68. The ready-to-use product according to any of claims 53 to 67, wherein the product comprises added lecithin or added phosphatidyl-choline.
